(19) United States
(12) Patent Application Publication
(10) Pub. No.: US 2017/0175121 A1
(43) Pub. Date: Jun. 22, 2017

(54) SELF ASSEMBLING NUCLEIC ACID NANOSTRUCTURES

(71) Applicant: Exicure, Inc., Skokie, IL (US)

(72) Inventor: Sergei Gryaznov, San Mateo, CA (US)

(73) Assignee: Exicure, Inc., Skokie, IL (US)

(21) Appl. No.: 15/301,467

(22) PCT Filed: Apr. 3, 2015
(86) PCT No.: PCT/US15/24255

§ 371 Date: Oct. 3, 2016

Related U.S. Application Data
(60) Provisional application No. 61/974,694, filed on Apr. 3, 2014.

(51) Int. Cl. C12N 15/117

(52) U.S. Cl. CPC C12N 15/117; C12N 2310/17; C12N 2310/151; C12N 2310/3515; C12N 2310/315; C12N 2310/3145

(57) ABSTRACT

Stable self-assembling nucleic acid nanostructures comprising: —a plurality of oligonucleotides, —a plurality of G-quadruplex forming nucleic acids linked to the plurality of oligonucleotides, and —a plurality of G-quadruplex stabilizing domains linked to the G-quadruplex forming nucleic acids. The nucleic acid nanostructures are suitable for use as agonists or antagonists of nucleic acid interacting complexes, such as Toll-like receptors; for inhibiting DNA or RNA expression; for stimulating or inhibiting an immune response; and for treating diseases such as cancer, infectious diseases, allergies and allergic diseases, and autoimmune diseases.

### RELATED APPLICATIONS

This application claims priority under 35 U.S.C. §119(e) to U.S. Provisional Application Ser. No. 61/974,694, entitled “SELF ASSEMBLING NUCLEIC ACID NANOSTRUCTURES” filed on Apr. 3, 2014, which is herein incorporated by reference in its entirety.

### FIELD OF INVENTION

The invention relates to self-assembling nucleic acid nanostructures, as well as methods and compositions thereof.

### BACKGROUND OF INVENTION

Existing therapies and vaccines fail to induce effective immune responses in a variety of diseases with critical worldwide impact, including AIDS, malaria, chlamydia, various malignancies and allergies or allergic diseases, such as asthma. Among the immunomodulatory compounds being developed, agonists of Toll-like receptors (TLR) have demonstrated outstanding potential. Agonists of TLR4, such as monophosphoryl lipid A (MPL) have reached late stages of clinical trials and approval in various countries in some instances. TLRs 7, 8, and 9 are all resident with the endosome of immune cells. TLR9 recognizes unmethylated CpG motifs that are common to bacterial DNA but not human DNA. TLRs 7 and 8 both recognize a specific sequence of short single stranded RNA common to viral infections. Importantly, agonists and antagonists of these common recognition motifs that can antagonize their respective TLRs and block or stimulate downstream signaling are known. However, their use in therapies is limited due to their ability to be delivered to the sites of pathology without being degraded in vivo. Concerns due to lack of efficacy, off-target phosphorothioate effects, and toxicity have slowed effective clinical translation of TLR 7/8 and 9 agonists and antagonists.

### SUMMARY OF INVENTION

Described herein are stable self-assembling nucleic acid nanostructures having a variety of uses. In some aspects the invention is a stable self-assembling nucleic acid nanostructure, comprising a plurality of oligonucleotides, wherein each internucleotide linkage of the oligonucleotide is not a phosphorothioate linkage, a plurality of G-quadruplex forming nucleic acids linked to the plurality of oligonucleotides, wherein the G-quadruplex forming nucleic acid is not TAGGGTT, and a plurality of G-quadruplex stabilizing domains linked to the G-quadruplex forming nucleic acids, wherein the oligonucleotides, the G-quadruplex forming nucleic acids and the G-quadruplex stabilizing domains form a plurality of G-quad structures. In some embodiments the self-assembling nucleic acid nanostructure does not have an inorganic core.

In other aspects the invention is a stable self-assembling nucleic acid nanostructure, comprising a plurality of oligonucleotides, a plurality of G-quadruplex forming nucleic acids linked to the plurality of oligonucleotides, wherein the G-quadruplex forming nucleic acids is not TAGGGTT, and a plurality of G-quadruplex stabilizing domains linked to the G-quadruplex forming nucleic acids, wherein when at least one of the G-quadruplex forming nucleic acids comprises GG, GGG, or GGGG and the oligonucleotide is CpG oligonucleotide the lipid is not diacyl lipid, wherein the oligonucleotides, the G-quadruplex forming nucleic acids and the G-quadruplex stabilizing domains form a plurality of G-quad structures. In some embodiments the self-assembling nucleic acid nanostructure does not have an inorganic core.

In some aspects the invention is a method for delivering a plurality of oligonucleotides to a subject by administering to the subject any of the self-assembling nucleic acid nanostructures described herein in order to deliver the oligonucleotides to the subject.

In other aspects the invention is a method for delivering a plurality of oligonucleotides to a subject by administering to a subject a stable self-assembling nucleic acid nanostructure, comprising a plurality of oligonucleotides, a plurality of G-quadruplex forming nucleic acids linked to the plurality of oligonucleotides, and a plurality of G-quadruplex stabilizing domains linked to the G-quadruplex forming nucleic acids, wherein the oligonucleotides, the G-quadruplex forming nucleic acids and the G-quadruplex stabilizing domains form a plurality of G-quad structures, and wherein the plurality of oligonucleotides is delivered to the subject. In some embodiments the self-assembling nucleic acid nanostructure does not have an inorganic core.

In some embodiments the plurality of oligonucleotides includes at least one therapeutic oligonucleotide. In other embodiments the subject has a disorder and wherein the method is a method for treating the disorder. Optionally, the disorder is cancer, infectious disease, allergy, asthma, neurodegenerative disease, disorders of the skin, disorders of the bone, autoimmune diseases, or optical disease.

In some embodiments the plurality of oligonucleotides comprises oligonucleotides having identical nucleotide sequences. In other embodiments the plurality of oligonucleotides comprises oligonucleotides having at least two different nucleotide sequences. For instance, the plurality of oligonucleotides may comprise oligonucleotides having at 2-10 different nucleotide sequences.

In some embodiments the plurality of G-quadruplex forming nucleic acids comprise G-quadruplex forming nucleic acids having identical nucleotide sequences. In other embodiments the plurality of G-quadruplex forming nucleic acids comprises G-quadruplex forming nucleic acids having at least two different nucleotide sequences.

In some embodiments the plurality of G-quadruplex stabilizing domains comprises identical G-quadruplex stabilizing domains. Optionally the plurality of G-quadruplex stabilizing domains may have at least two different G-quadruplex stabilizing domains.

The nanostructure includes nucleic acids which may or may not have modified internucleotide linkages or bases or sugars. For instance in some embodiments each internucleotide linkage of the oligonucleotide, nucleic acid, or G-quadruplex stabilizing domain is or is not a phosphorothioate linkage. In other embodiments at least one internucleotide linkage of the o oligonucleotide, nucleic acid, or G-quadruplex forming nucleic acid is a phosphorothioate linkage. In yet other embodiments at least one or each internucleotide linkage of the oligonucleotide, nucleic acid, or G-quadruplex forming nucleic acid is selected from a N3′-P5′ phosphoramidate linkage and a N3′-P5′thio-phosphoramidate linkage.

The thermodynamic stability of the nanostructure in some embodiments is high enough to provide for the overall structural stability of constructs under physiological salt and temperature conditions.

In some embodiments at least one of the oligonucleotides has 5′ termini exposed to the outside surface of the nano structure.

The nanostructure may include a therapeutic oligonucleotide. For instance the therapeutic oligonucleotide may be a CpG-group containing oligonucleotide, referred to as a CpG oligonucleotide. CpG oligonucleotides include, for instance, A-class, B-class and C-class CpG oligonucleotides. In some embodiments the plurality of oligonucleotides comprises RNA or antisense oligonucleotides. In other embodiments the plurality of oligonucleotides comprises TLR7 antagonists, TLR8 antagonists, TLR9 antagonists, TLR7 agonists, TLR8 agonists, or TLR9 agonists.

In some embodiments the plurality of G-quadruplex forming nucleic acids are

TTGGGGTT,
TAGGGTT,
(SEQ ID NO: 1)
GGTTGGTGTGGTTGG,
(SEQ ID NO: 2)
GGGTTTTGGG,
TTAGGG,
or
(SEQ ID NO: 3)
GGTGGTGGTGGTTGTGGTGGTGGTGG.

In other embodiments the plurality of G-quadruplex stabilizing domains is selected from the group consisting of diacyl lipids, monoacyl lipids, palmitic acid, stearic acid, cationic porphyrin, TMPyP4, small-molecule inhibitors of Telomerase, Telomestatin (SOT-095), anionic porphyrin N-methyl mesoporphyrin (NMM), ibenzophenanthrolines, 3,4-TMPyPz, Triaminoacridine derivatives, RHPS4, Isoalloxazines, and Se2SAP.

The linkage between the plurality of oligonucleotides and the plurality of G-quadruplex forming nucleic acids may be a covalent linkage.

The linkage between the plurality of G-quadruplex forming nucleic acids and the plurality of G-quadruplex stabilizing domains may be a covalent linkage.

In some embodiments the nanostructure further includes a linker connecting the plurality of oligonucleotides and the plurality of G-quadruplex forming nucleic acids. In some embodiments the linker is selected from the group consisting of HEG and PEG.

In some embodiments the nanostructures have an oligonucleotide surface density of at least 0.3 pmol/cm².

In other embodiments the nanostructure includes an antigen and optionally the surface density of the antigen is at least 0.3 pmol/cm². In other embodiments the antigen includes at least two different types of antigen.

In other embodiments the oligonucleotide is a therapeutic oligonucleotide and is RNA or DNA or a combination thereof. The oligonucleotides may be, for instance, a double stranded RNA, such as poly(I:C), a single stranded RNA such as an RNA containing UUG-motifs, or an unmethylated deoxyribonucleic acid, such as a CpG oligonucleotide.

In certain embodiments, the diameter of the nanostructure is from 1 nm to about 250 nm in mean diameter, about 1 ran to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

A vaccine composed of a nanostructure described herein and a carrier is provided according to other aspects of the invention.

A method for delivering a therapeutic agent to a cell by delivering the nanostructure of the invention to the cell is provided in other aspects.

A method for regulating expression of a target molecule is provided in other aspects of the invention. The method involves delivering the nanostructure of the invention to the cell. In some embodiments the target molecule is a TLR selected from the group consisting of TLR 7, 8, and 9.

A method for activating a TLR by delivering the nanostructure as described herein to the cell is provided in other aspects of the invention.

A method for inhibiting a TLR by delivering the nanostructure as described herein to the cell is provided in other aspects of the invention.

According to other aspects the invention is a method of treating a subject, involving administering to the subject the nanostructure as described herein in an effective amount to stimulate an immune response. In some embodiments the subject has an infectious disease, a cancer, an autoimmune disease, allergy, or an allergic disease such as asthma.

Further aspects of the invention relate to a kit comprising a nanostructure as described herein. In certain embodiments, the kit further comprises instructions for use.

Other aspects of the invention relate to compositions for use in the treatment of disease. The compositions include any of the stable self-assembling nucleic acid nanostructures described herein.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C show a non-limiting example of a minimal building block of a nanostructure of the invention. 1A. A general structure of the minimal building block including two oligonucleotides, two G-quadruplex stabilizing domains with linkers and a single G-quadruplex forming nucleic acid. 1B. A general structure having the addition of a second G-quadruplex forming nucleic acid. 1C. Chemical structure of two units of a G-quadruplex nucleic acid.

FIG. 2 is a schematic depicting several examples of the nanostructure of the invention.

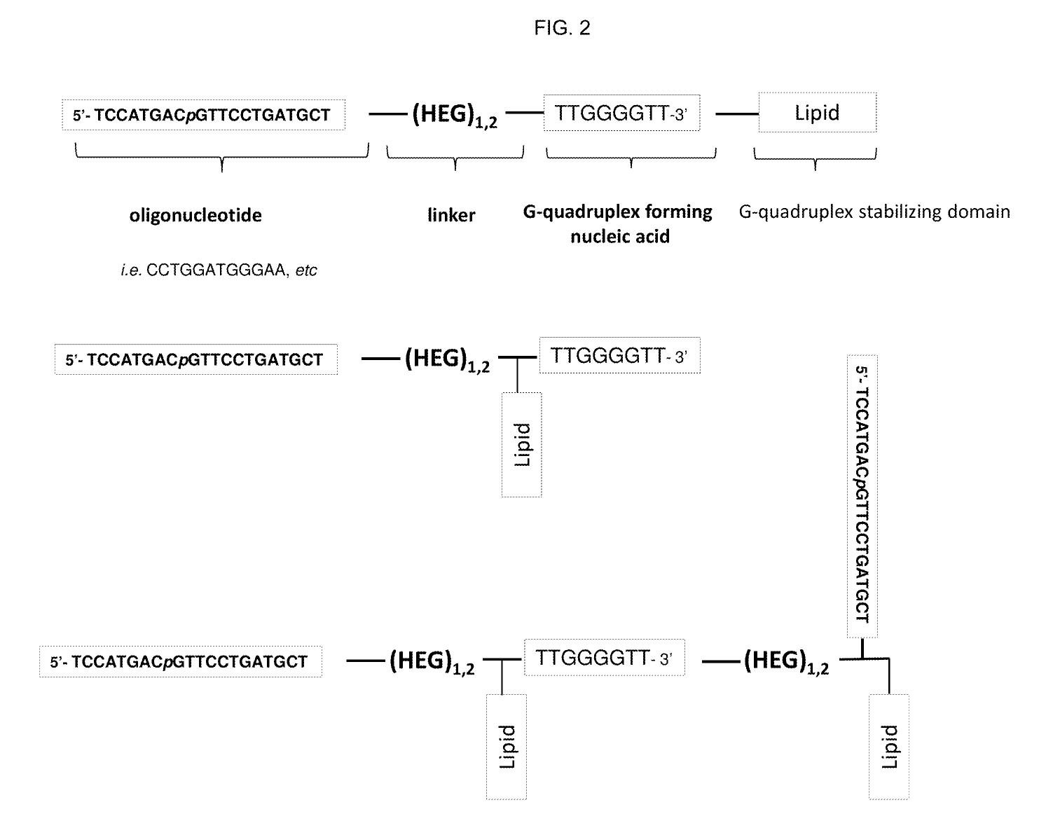

TCCATGACGTTCCTGATGCT is SEQ ID NO: 88
and
CCTGGATGGGAA is SEQ ID NO: 89.

FIGS. 3A-3B depict several examples of components of the nanostructure of the invention. 3A depicts an exemplary set of four G-quadruplex stabilizing domains linked to 4 G-quadruplex nucleic acids. 3B depicts a single guanine base versus stable G-quadruplex.

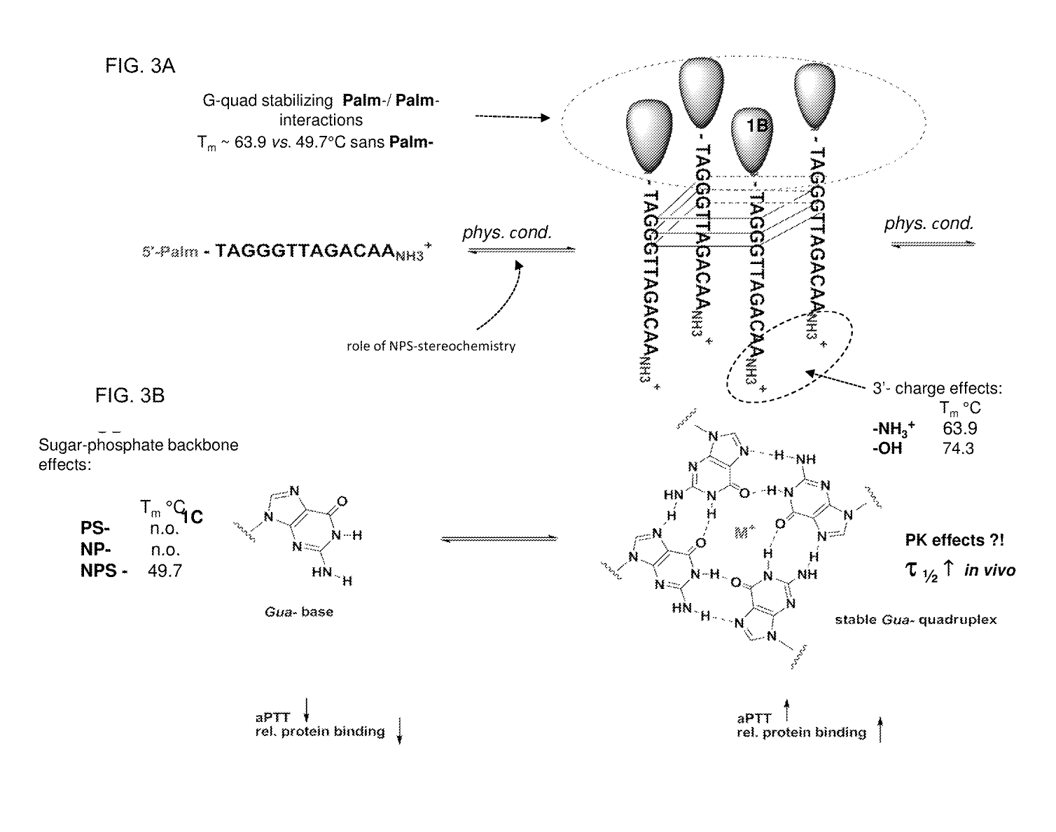

TAGGGTTAGACAA is SEQ ID NO: 90.

FIGS. 4A-4B show a set of structures to depict various examples of G-quadruplexes. 4A is a chemical structure depicting the interactions between 4 Gs to form a G-quadruplex. 4B provides 5 exemplary sequences and the three dimensional shape of the corresponding G-quadruplex.

GGTTGGTGTGGTTGG is SEQ ID NO: 1,
GGTGGTGGTGGTTGTGGTGGTGGTGG is SEQ ID NO: 91
and
GGGTTTTGGG is SEQ ID NO: 92.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The present invention, in some aspects, overcomes several major hurdles encountered by conventional nucleic acid delivery mechanisms by achieving greater efficacy, lower in vivo toxicity, favorable PK/PD properties, such as, no long term accumulation in major organs, traceable CMC characteristics, faster activation, changing cellular distribution, and facilitating simple and scalable synthesis of various combinations of therapeutics including combinations of nucleic acids, adjuvant and antigen-containing structures, among others. The nanostructures of the invention result in more effective therapies for prophylactic or therapeutic uses in treating a wide variety of diseases/infections including, for example, AIDS, malaria, chlamydia, campylobacter, cytomegalovirus, dengue, Epstein-Ban mononucleosis, foot and mouth disease, rabies, Helicobacter pylori gastric ulcers, hepatitis A, B, C, herpes simplex, influenza, leishmaniasis, cholera, diphtheria, Haemophilus influenza, meningococcal meningitis, plague, pneumococcal pneumonia, tetanus, typhoid fever, respiratory synctial virus, rhinovirus, schistosomiasis, shigella, streptococcus group A and B, tuberculosis, vibrio cholera, salmonella, aspergillus, blastomyces, histoplasma, candida, cryptococcus, pneumocystis, and urinary tract infections; various food allergies such as peanut, fruit, garlic, oats, meat, milk, fish, shellfish, soy, tree nut, wheat, gluten, egg, sulphites; various drug allergies such as to tetracycline, Dilantin, carbamazepine, penicillins, cephalosporins, sulfonamides, NSAIDs, intravenous contrast dye, local anesthetics; autoimmune diseases such as multiple sclerosis, lupus, inflammatory bowel disease, Crohn's disease, ulcerative colitis, asthma, and COPD; and cancers such as melanoma, breast cancer, prostate cancer, bladder cancer, NSCLC, glioblastoma multiforme, among others.

The nanostructures of the invention are stable self-assembling nucleic acid nanostructures that in preferred embodiments do not have an inorganic core. An inorganic core refers to a central or core component of the structure. Typical inorganic core materials include but are not limited to gold, silver, platinum, aluminum, palladium, copper, cobalt, indium, nickel and mixtures thereof. In some instances the nanostructure is free of inorganic material.

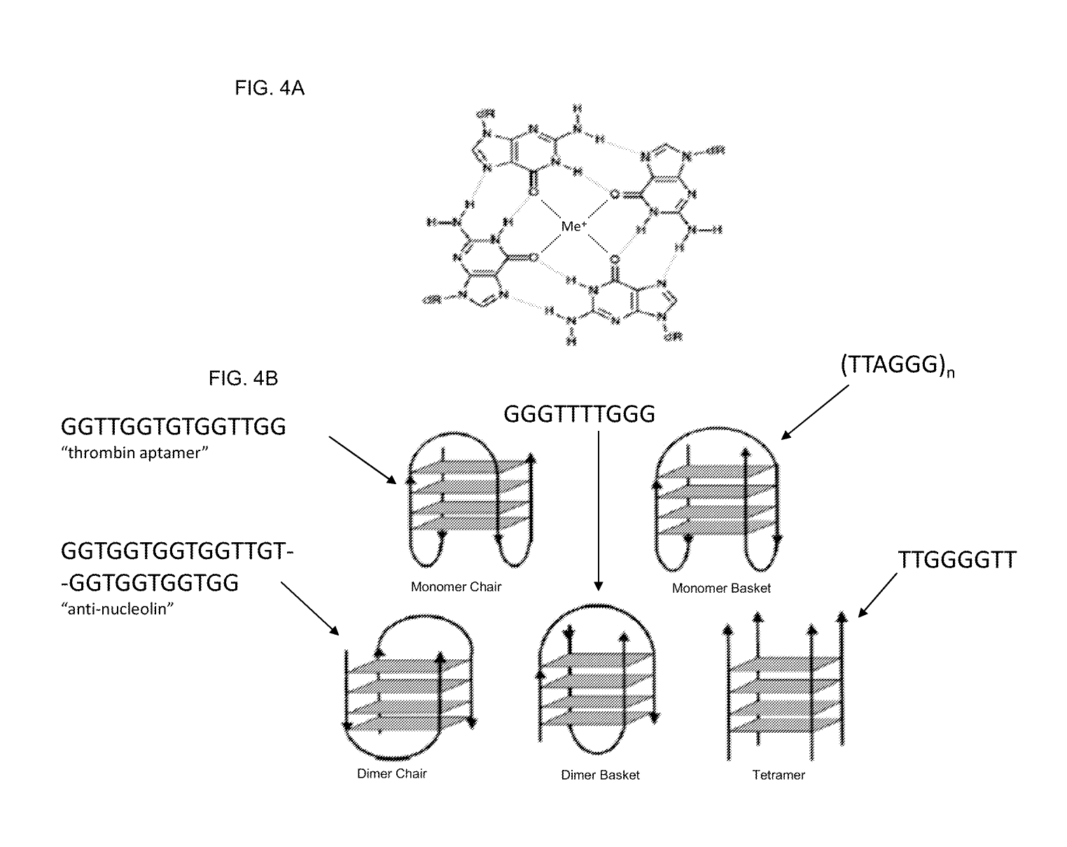

The nanostructures are composed of a minimum of two components: a plurality of oligonucleotides and a plurality of G-quadruplex forming nucleic acid linked to the plurality of oligonucleotides. A third element which may be included in the nanostructure is a G-quadruplex stabilizing domain. The core elements self-assemble in a manner that results in the formation of a G-quad structure. A G-quad structure is a three dimensional arrangement of each of the elements based primarily on the interactions between the Gs of the G-quadruplex forming nucleic acids. Some exemplary G-quad structures are shown in FIG. 4B. These include a monomer chair, a monomer basket, a dimer chair, a dimer basket, and a tetramer.

The G-quad structure results in a set of nucleic acids arranged in a geometric shape, i.e. a 3-dimensionally shaped layer of nucleic acids, potentially with outwardly facing therapeutic oligonucleotides. The outwardly facing oligonucleotides may have exposed 5′ ends or 3′ ends or a mixture thereof. The degree to which the end of the oligonucleotide is outwardly facing and thus exposed to the physiological environment can be controlled by direction in which the oligonucleotide is linked to the G-quadruplex forming nucleic acid. In some instances it is desirable to have more oligonucleotides having an exposed 5′end than a 3′end. For instance, a nanostructure may have at least 50%, 60%, 70%, 80%, 90% or 95% of the oligonucleotides having an exposed 5′end. In some instances the oligonucleotide may be two oligonucleotides linked together at the 3′ ends.

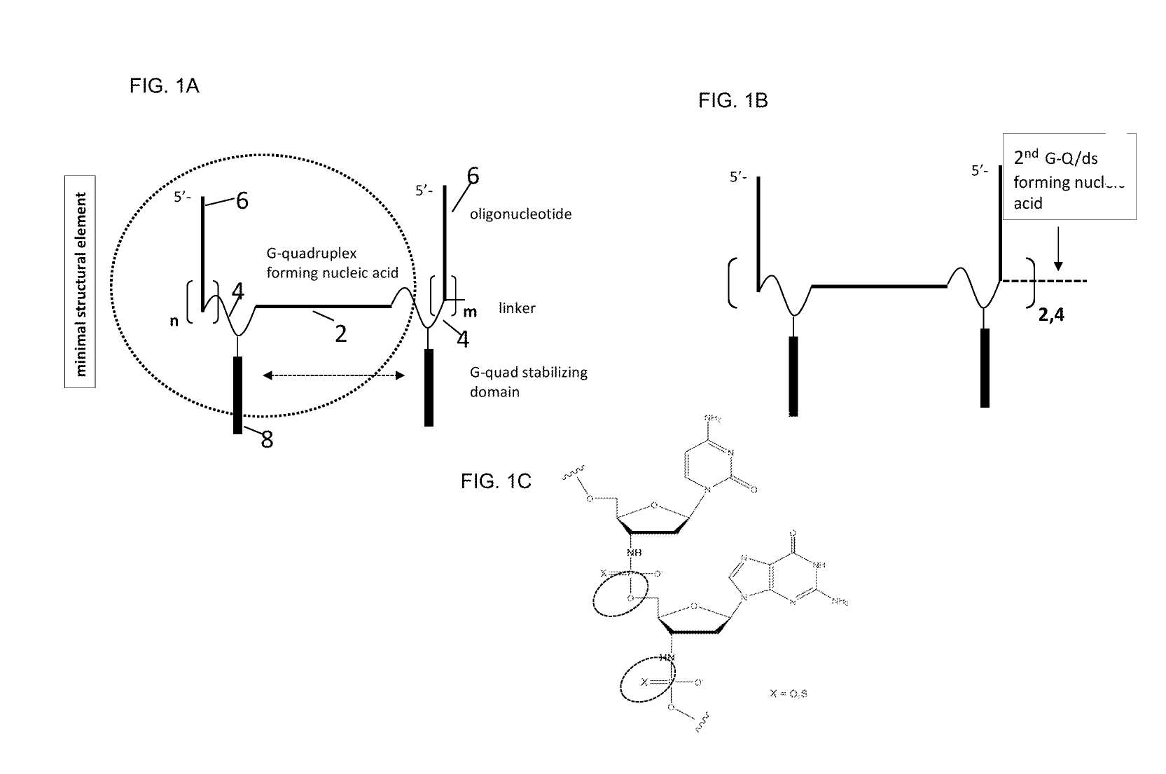

A set of exemplary building blocks of the nanostructures of the invention is shown in the schematic of FIG. 1. A centrally positioned G-quadruplex forming nucleic acid (2) is shown in FIG. 1A having a linker (4) at either end. Each linker is connected to an oligonucleotide (6). A G-quadruplex forming nucleic acid, as used herein, is a G-rich oligonucleotide which is capable of forming with other G-quadruplex forming nucleic acids, referred to as a G-quad. The G-quadruplex forming nucleic acids may have a sequence that includes at least 50% G's. In some embodiment the G-quadruplex forming nucleic acids have a sequence that includes at least 60%, 70%, or 80% G's. The G-quadruplex forming nucleic acids may have a sequence that includes all G's and T's. In some embodiments it has a sequence of at least 60%, 70%, 80% or 90% G's and T's. The G-quadruplex forming nucleic acids may also have one or multiple G repeats. For instance, a G-quadruplex forming nucleic acid may have a stretch of at least 4 G's. In other embodiments the G-quadruplex forming nucleic acid may have one or more stretches of 3 G's. In yet other embodiments the G-quadruplex forming nucleic acid may have multiple G dimers (e.g., 2, 3, 4, 5, or 6 dimers) separated by one or more other nucleotides, such as 1, 2, or 3 T's.

A nanostructure of the invention may be composed of multiple units having the same G-quadruplex forming nucleic acid or alternatively may have two or more different types of G-quadruplex forming nucleic acids. The three dimensional structure of the nanostructure may be altered by using identical or a mixture of different G-quadruplex forming nucleic acids. The structure of the G-quad is dependent to a significant effect on the specific hydrogen bond formation resulting from the G's in the G-quadruplex forming nucleic acids. The ability to form stable hydrogen bonds can be altered by manipulating the G-chemistry, which can be accomplished in a variety of ways. For instance modifications can be made to the nucleotide or to the internucleotide linkage. The G-quadruplex forming nucleic acids may have a phosphodiester internucleotide linkage or a modified internucleotide linkage. The modified internucleotide linkage includes but is not limited to phosphorothioate linkages, phosphoramidated linkages, and/or thiophosphoramidate linkages. The use of phosphoramidated linkages, and/or thiophosphoramidate linkages in the G-quadruplex forming nucleic acids helps enhance the thermodynamic stability of the G-quad.

The structure of a phosphodiester versus a phosphoramidated internucleotide linkage for RNA and DNA is shown below.

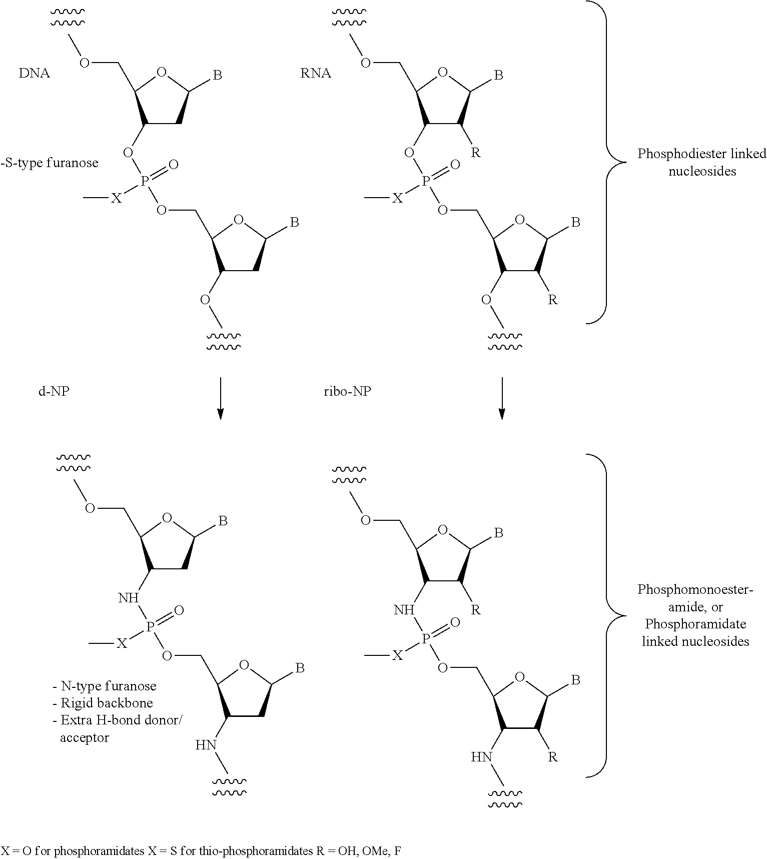

It is desirable to enhance the thermodynamic stability of the nanostructure, such that it can withstand in vivo physiological salt and temperature conditions. Unlike many other nucleic acid delivery devices which are designed for rapid degradation and release of oligonucleotide upon delivery in vivo, the nanostructures of the invention are designed to withstand physiological conditions, such that the oligonucleotides remain attached to the structure when exposed to the tissue. In addition to manipulating the G-chemistry in order to enhance thermodynamic stability, stabilizing groups referred to herein as G-quadruplex stabilizing domains may be linked to the G-quadruplex forming nucleic acids. G-quadruplex stabilizing domains also enhance thermodynamic stability.

A “G-quadruplex stabilizing domain” as used herein refers to a lipid containing or micelle forming structure that when linked to the G-quadruplex forming nucleic acids enhances the stability of the G-quad. G-quadruplex stabilizing domains include but are not limited to diacyl lipids, monoacyl lipids, palmitic acid, stearic acid, cationic porphyrin, TMPyP4, small-molecule inhibitors of Telomerase, Telomestatin (SOT-095), anionic porphyrin N-methyl mesoporphyrin (NMM), ibenzophenanthrolines, 3,4-TMPyPz, Triaminoacridine derivatives, RHPS4, Isoalloxazines, and Se2SAP. In addition to enhancing the thermodynamic stability of the nanostructure, the G-quadruplex stabilizing domains also have an impact on the degree of multimerization.

Some or all of the nucleic acids and stabilizing domains of the nanostructure may be linked to one another either directly or indirectly through a covalent or non-covalent linkage. The linkage of one nucleic acid to another nucleic acid may be in addition to or alternatively to the linkage of that nucleic acid to a stabilizing domain. One or more of the nucleic acids or the stabilizing domain may also be linked to other molecules such as an antigen. The nucleic acids may be linked to one another either directly or indirectly through a covalent or non-covalent linkage.

The nanostructure of the invention also includes an oligonucleotide which is preferably a therapeutic oligonucleotide. An oligonucleotide, as used herein, refers to any nucleic acid containing molecule. The nucleic acid may be DNA, RNA, PNA, LNA, ENA or combinations or modifications thereof. It may also be single, double or triple stranded. A therapeutic oligonucleotide is an oligonucleotide that can function as a therapeutic and or diagnostic agent.

Therapeutic oligonucleotides include but are not limited to immunomodulatory oligonucleotides, inhibitory oligonucleotides, expression enhancing oligonucleotides and diagnostic oligonucleotides.

In some embodiments the immunomodulatory oligonucleotide is a TLR agonist or antagonist. A TLR agonist, as used herein is a nucleic acid molecule that interacts with and stimulates the activity of a TLR. A TLR antagonist, as used herein, is a nucleic acid molecule that interacts with and modulates, i.e. reduces, the activity of a TLR.

Toll-like receptors (TLRs) are a family of highly conserved polypeptides that play a critical role in innate immunity in mammals. At least ten family members, designated TLR1-TLR10, have been identified. The cytoplasmic domains of the various TLRs are characterized by a Toll-interleukin 1 (IL-1) receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adaptor protein MyD88 has been reported to associate with TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines. For a review, see Aderem A et al. (2000) Nature 406:782-87.

TLRs are believed to be differentially expressed in various tissues and on various types of immune cells. For example, human TLR7 has been reported to be expressed in placenta, lung, spleen, lymph nodes, tonsil and on plasmacytoid precursor dendritic cells (pDCs). Chuang T-H et al. (2000) Eur Cytokine Netw 11:372-8); Kadowaki N et al. (2001) J Exp Med 194:863-9. Human TLR8 has been reported to be expressed in lung, peripheral blood leukocytes (PBL), placenta, spleen, lymph nodes, and on monocytes. Kadowaki N et al. (2001) J Exp Med 194:863-9; Chuang T-H et al. (2000) Eur Cytokine Netw 11:372-8. Human TLR9 is reportedly expressed in spleen, lymph nodes, bone marrow, PBL, and on pDCs, and B cells. Kadowaki N et al. (2001) J Exp Med 194:863-9; Bauer S et al. (2001) Proc Natl Acad Sci USA 98:9237-42; Chuang T-H et al. (2000) Eur Cytokine Netw 11:372-8.

Nucleotide and amino acid sequences of human and murine TLR7 are known. See, for example, GenBank Accession Nos. AF240467, AF245702, NM_016562, AF334942, NM_133211; and AAF60188, AAF78035, NP_057646, AAL73191, and AAL73192, the contents of all of which are incorporated herein by reference. Human TLR7 is reported to be 1049 amino acids long. Murine TLR7 is reported to be 1050 amino acids long. TLR7 polypeptides include an extracellular domain having a leucine-rich repeat region, a transmembrane domain, and an intracellular domain that includes a TIR domain.

Nucleotide and amino acid sequences of human and murine TLR8 are known. See, for example, GenBank Accession Nos. AF246971, AF245703, NM_016610, XM_045706, AY035890, NM_133212; and AAF64061, AAF78036, NP_057694, XP_045706, AAK62677, and NP_573475, the contents of all of which is incorporated herein by reference. Human TLR8 is reported to exist in at least two isoforms, one 1041 amino acids long and the other 1059 amino acids long. Murine TLR8 is 1032 amino acids long. TLR8 polypeptides include an extracellular domain having a leucine-rich repeat region, a transmembrane domain, and an intracellular domain that includes a TIR domain.

Nucleotide and amino acid sequences of human and murine TLR9 are known. See, for example, GenBank Accession Nos. NM_017442, AF259262, AB045180, AF245704, AB045181, AF348140, AF314224, NM_031178; and NP_059138, AAF72189, BAB19259, AAF78037, BAB19260, AAK29625, AAK28488, and NP_112455, the contents of all of which are incorporated herein by reference. Human TLR9 is reported to exist in at least two isoforms, one 1032 amino acids long and the other 1055 amino acids. Murine TLR9 is 1032 amino acids long. TLR9 polypeptides include an extracellular domain having a leucine-rich repeat region, a transmembrane domain, and an intracellular domain that includes a TIR domain.

As used herein, the term “TLR signaling” refers to any aspect of intracellular signaling associated with signaling through a TLR. As used herein, the term “TLR-mediated immune response” refers to the immune response that is associated with TLR signaling. A reduction in TLR signaling or activity refers to a decrease in signaling or activity relative to baseline. A baseline level may be a level where an immunostimulatory molecule is causing stimulation of a TLR. In that instance a reduction in signaling or activity is a reduction in signaling or activity with respect to the level of signaling or activity achieved by the immunostimulatory molecule.

A TLR7-mediated immune response is a response associated with TLR7 signaling. TLR7-mediated immune response is generally characterized by the induction of IFN-α and IFN-inducible cytokines such as IP-10 and I-TAC. The levels of cytokines IL-1 α/β, IL-6, IL-8, MIP-1α/β and MIP-3α/β induced in a TLR7-mediated immune response are less than those induced in a TLR8-mediated immune response.

A TLR8-mediated immune response is a response associated with TLR8 signaling. This response is further characterized by the induction of pro-inflammatory cytokines such as IFN-γ, IL-12p40/70, TNF-α, IL-1α/β, IL-6, IL-8, MIP-1 α/β and MIP-3 α/β.

A TLR9-mediated immune response is a response associated with TLR9 signaling. This response is further characterized at least by the production/secretion of IFN-γ and IL-12, albeit at levels lower than are achieved via a TLR8-mediated immune response.

As used herein, a “TLR7/8 agonist” collectively refers to any nucleic acid that is capable of increasing TLR7 and/or TLR8 signaling (i.e., an agonist of TLR7 and/or TLR8). Some TLR7/8 ligands induce TLR7 signaling alone (e.g., TLR7 specific agonists), some induce TLR8 signaling alone (e.g., TLR8 specific agonists), and others induce both TLR7 and TLR8 signaling.

The level of TLR7 or TLR8 signaling may be enhanced over a pre-existing level of signaling or it may be induced over a background level of signaling. TLR7 ligands include, without limitation, guanosine analogues such as C8-substituted guanosines, mixtures of ribonucleosides consisting essentially of G and U, guanosine ribonucleotides and RNA or RNA-like molecules (PCT/US03/10406), and adenosine-based compounds (e.g., 6-amino-9-benzyl-2-(3-hydroxy-propoxy)-9H-purin-8-ol, and similar compounds made by Sumitomo (e.g., CL-029)).

As used herein, the term “guanosine analogues” refers to a guanosine-like nucleotide (excluding guanosine) having a chemical modification involving the guanine base, guanosine nucleoside sugar, or both the guanine base and the guanosine nucleoside sugar. Guanosine analogues specifically include, without limitation, 7-deaza-guanosine.

Guanosine analogues further include C8-substituted guanosines such as 7-thia-8-oxoguanosine (immunosine), 8-mercaptoguanosine, 8-bromoguanosine, 8-methylguanosine, 8-oxo-7,8-dihydroguanosine, C8-arylamino-2′-deoxyguanosine, C8-propynyl-guanosine, C8- and N7-substituted guanine ribonucleosides such as 7-allyl-8-oxoguanosine (loxoribine) and 7-methyl-8-oxoguanosine, 8-aminoguanosine, 8-hydroxy-2′-deoxyguanosine, 8-hydroxyguanosine, and 7-deaza 8-substituted guanosine.

TLR8 ligands include mixtures of ribonucleosides consisting essentially of G and U, guanosine ribonucleotides and RNA or RNA-like molecules (PCT/US03/10406). Additional TLR8 ligands are also disclosed in Gorden et al. J. Immunol. 2005, 174:1259-1268).

As used herein, the term “TLR9 agonist” refers to any agent that is capable of increasing TLR9 signaling (i.e., an agonist of TLR9). TLR9 agonists specifically include, without limitation, immunostimulatory nucleic acids, and in particular CpG immunostimulatory nucleic acids.

An “immunostimulatory oligonucleotide” as used herein is any nucleic acid (DNA or RNA) containing an immunostimulatory motif or backbone that is capable of inducing an immune response. An induction of an immune response refers to any increase in number or activity of an immune cell, or an increase in expression or absolute levels of an immune factor, such as a cytokine. Immune cells include, but are not limited to, NK cells, CD4+ T lymphocytes, CD8+ T lymphocytes, B cells, dendritic cells, macrophage and other antigen-presenting cells. Cytokines include, but are not limited to, interleukins, TNF-α, IFN-α,β and γ, Flt-ligand, and co-stimulatory molecules. Immunostimulatory motifs include, but are not limited to CpG motifs and T-rich motifs.

The immunostimulatory oligonucleotides of the nanoscale construct are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

As used herein, the term “immunostimulatory CpG nucleic acids” or “immunostimulatory CpG oligonucleotides” refers to any CpG-containing nucleic acid that is capable of activating an immune cell. At least the C of the CpG dinucleotide is typically, but not necessarily, unmethylated. Immunostimulatory CpG nucleic acids are described in a number of issued patents and published patent applications, including U.S. Pat. Nos. 6,194,388; 6,207,646; 6,218,371; 6,239,116; 6,339,068; 6,406,705; and 6,429,199.

In some embodiments the immunostimulatory oligonucleotides have a modified backbone such as a phosphorothioate (PS) backbone. In other embodiments the immunostimulatory oligonucleotides have a phosphodiester (PO) backbone. In yet other embodiments immunostimulatory oligonucleotides have a mixed PO and PS backbone.

A non-limiting set of immunostimulatory oligonucleotides includes:

dsRNA (TLR 3): poly(A:U) and poly(I:C)
ssRNA (TLR7/8):
(SEQ ID NO: 4)
CCGUCUGUUGUGUGACUC
(SEQ ID NO: 5)
GCCACCGAGCCGAAGGCACC
(SEQ ID NO: 6)
UAUAUAUAUAUAUAUAUAUA
(SEQ ID NO: 7)
UUAUUAUUAUUAUUAUUAUU
(SEQ ID NO: 8)
UUUUAUUUUAUUUUAUUUUA
(SEQ ID NO: 9)
UGUGUGUGUGUGUGUGUGUG
(SEQ ID NO: 10)
UUGUUGUUGUUGUUGUUGUU
(SEQ ID NO: 11)
UUUGUUUGUUUGUUUGUUUG
(SEQ ID NO: 12)
UUAUUUAUUUAUUUAUUUAUUUAU
(SEQ ID NO: 13)
UUGUUUGUUUGUUUGUUUGUUUGU
(SEQ ID NO: 14)
GCCCGUCUGUUGUGUGACUC
(SEQ ID NO: 15)
GUCCUUCAAGUCCUUCAA
DNA (TLR9):
(SEQ ID NO: 16)
GGTGCATCGATGCAGGGGGG
(SEQ ID NO: 17)
TCCATGGACGTTCCTGAGCGTT
(SEQ ID NO: 18)
TCGTCGTTCGAACGACGTTGAT
(SEQ ID NO: 19)
TCGTCGACGATCCGCGCGCGCG
(SEQ ID NO: 20)
GGGGTCAACGTTGAGGGGGG
(SEQ ID NO: 21)
TCGTCGTTTTGTCGTTTTGTCGTT
(SEQ ID NO: 22)
TCGTCGTTGTCGTTTTGTCGTT
(SEQ ID NO: 23)
GGGGGACGATCGTCGGGGGG
(SEQ ID NO: 24)
GGGGACGACGTCGTGGGGGGG
(SEQ ID NO: 25)
TCGTCGTTTTCGGCGCGCGCCG
(SEQ ID NO: 26)
TCGTCGTCGTTCGAACGACGTTGAT

As used herein, a “TLR7/8 antagonist” collectively refers to any nucleic acid that is capable of decreasing TLR7 and/or TLR8 signaling (i.e., an antagonist of TLR7 and/or TLR8) relative to a baseline level. Some TLR7/8 antagonists decrease TLR7 signaling alone (e.g., TLR7 specific antagonists), some decrease TLR8 signaling alone (e.g., TLR8 specific antagonists), and others decrease both TLR7 and TLR8 signaling.

As used herein, the term “TLR9 antagonist” refers to any agent that is capable of decreasing TLR9 signaling (i.e., an antagonist of TLR9).

In some embodiments antagonists of TLR 7,8, or 9 include immunoregulatory nucleic acids. Immunoregulatory nucleic acids include but are not limited to nucleic acids falling within the following formulas: 5′RₙJGCN_{z}3′, wherein each R is a nucleotide, n is an integer from about 0 to 10, J is U or T, each N is a nucleotide, and z is an integer from about 1 to about 100. In some embodiments, ₙ is 0 and _{z} is from about 1 to about 50. In some embodiments N is 5'S₁S₂S₃S₄3′, wherein S₁, S₂, S₃, and S₄ are independently G, I, or 7-deaza-dG. In some embodiments the TLR7 TLR8 and/or TLR9 antagonist is selected from the group consisting of

(SEQ ID NO: 27)
TCCTGGAGGGGTTGT,
(SEQ ID NO: 28)
TGCTCCTGGAGGGGTTGT,
(SEQ ID NO: 29)
TGCTGGATGGGAA,
(SEQ ID NO: 30)
TGCCCTGGATGGGAA,
(SEQ ID NO: 31)
TGCTTGACACCTGGATGGGAA,
(SEQ ID NO: 32)
TGCTGGATGGGAA/iSp18//iSp18//,
(SEQ ID NO: 33)
TGCCCTGGATGGGAA/iSp18//iSp18//,
(SEQ ID NO: 34)
TGCTTGACACCTGGATGGGAA/iSp18//iSp18//,
(SEQ ID NO: 35)
TCCTGAGCTTGAAGT/iSp18//iSp18/,
(SEQ ID NO: 36)
TCCTGAGCTTGAAGT/iSp18//iSp18//,
(SEQ ID NO: 37)
TTCTGGCGGGGAAGT/iSp18//iSp18/,
(SEQ ID NO: 38)
CTCCTATTGGGGGTTTCCTAT/iSp18//iSp18/,
(SEQ ID NO: 39)
ACCCCCTCTACCCCCTCTACCCCTCT/iSp18//iSp18/,
(SEQ ID NO: 40)
CCTGGATGGGAA/iSp18//iSp18/,
(SEQ ID NO: 41)
TTCTGGCGGGGAAGT/iSp18//iSp18//,
(SEQ ID NO: 42)
CTCCTATTGGGGGTTTCCTAT/iSp18//iSp18//,
(SEQ ID NO: 43)
ACCCCCTCTACCCCCTCTACCCCTCT/iSp18//iSp18//,
(SEQ ID NO: 44)
CCTGGATGGGAA/iSp18//iSp18//,
(SEQ ID NO: 40)
C*C*T*GGATGGGAA/iSp18//iSp18//,
(SEQ ID NO: 40)
CCTGGATG*G*G*AA/iSp18//iSp18//,
(SEQ ID NO: 40)
C*C*T*GGATG*G*G*AA/iSp18//iSp18//,
(SEQ ID NO: 45)
/Chol/CCTGGATGGGAA/iSp18//iSp18//,
(SEQ ID NO: 46)
/Stryl/CCTGGATGGGAA/iSp18//iSp18//,
(SEQ ID NO: 47)
/Palm/CCTGGATGGGAA/iSp18//iSp18//,
(SEQ ID NO: 27)
T*C*C*T*G*G*A*G*G*G*G*T*T*G*T
(SEQ ID NO: 28)
T*G*C*T*C*C*T*G*G*A*G*G*G*G*T*T*G*T
(SEQ ID NO: 29)
T*G*C*T*G*G*A*T*G*G*G*A*A
(SEQ ID NO: 30)
T*G*C*C*C*T*G*G*A*T*G*G*G*A*A
(SEQ ID NO: 31)
T*G*C*T*T*G*A*C*A*C*C*T*G*G*A*T*G*G*G*A*A
(SEQ ID NO: 32)
T*G*C*T*G*G*A*T*G*G*G*A*A*/iSp18//iSp18//
(SEQ ID NO: 33)
T*G*C*C*C*T*G*G*A*T*G*G*G*A*A*/iSp18//iSp18//
(SEQ ID NO: 34)
T*G*C*T*T*G*A*C*A*C*C*T*G*G*A*T*G*G*G*A*A*/
iSp18//iSp18//
(SEQ ID NO: 35)
T*C*C*T*G*A*G*C*T*T*G*A*A*G*T*/iSp18//iSp18//
(SEQ ID NO: 36)
T*C*C*T*G*A*G*C*T*T*G*A*A*G*T*/iSp18//iSp18//
(SEQ ID NO: 37)
T*T*C*T*G*G*C*G*G*G*G*A*A*G*T*/iSp18//iSp18//
(SEQ ID NO: 38)
C*T*C*C*T*A*T*T*G*G*G*G*G*T*T*T*C*C*T*A*T*/
iSp18//iSp18//
(SEQ ID NO: 39)
A*C*C*C*C*C*T*C*T*A*C*C*C*C*C*T*C*T*A*C*C*C*C*T*
C*T*/iSp18//iSp18//
(SEQ ID NO: 40)
C*C*T*G*G*A*T*G*G*G*A*A*/iSp18//iSp18//
(SEQ ID NO: 41)
T*T*C*T*G*G*C*G*G*G*G*A*A*G*T*/iSp18//iSp18//
(SEQ ID NO: 42)
C*T*C*C*T*A*T*T*G*G*G*G*G*T*T*T*C*C*T*A*T*/
iSp18//iSp18//
(SEQ ID NO: 43)
A*C*C*C*C*C*T*C*T*A*C*C*C*C*C*T*C*T*A*C*C*C*C*T*
C*T*/iSp18//iSp18//
(SEQ ID NO: 44)
C*C*T*G*G*A*T*G*G*G*A*A*/iSp18//iSp18//
(SEQ ID NO: 48)
/iSp18//iSp18/*T*G*C*T*G*G*A*T*G*G*G*A*A
(SEQ ID NO: 49)
/iSp18//iSp18/*T*G*C*C*C*T*G*G*A*T*G*G*G*A*A
(SEQ ID NO: 50)
/iSp18//iSp18/*T*G*C*T*T*G*A*C*A*C*C*T*G*G*A*T*G*
G*G*A*A
(SEQ ID NO: 51)
/iSp18//iSp18/*T*C*C*T*G*A*G*C*T*T*G*A*A*G*T
(SEQ ID NO: 52)
/iSp18//iSp18/*T*C*C*T*G*A*G*C*T*T*G*A*A*G*T
(SEQ ID NO: 53)
/iSp18//iSp18/*T*T*C*T*G*G*C*G*G*G*G*A*A*G*T
(SEQ ID NO: 54)
/iSp18//iSp18/*C*T*C*C*T*A*T*T*G*G*G*G*G*T*T*T*C*
C*T*A*T
(SEQ ID NO: 55)
/iSp18//iSp18/*A*C*C*C*C*C*T*C*T*A*C*C*C*C*C*T*C*
T*A*C*C*C*C*T*C*T
(SEQ ID NO: 56)
/iSp18//iSp18/*C*C*T*G*G*A*T*G*G*G*A*A
(SEQ ID NO: 57)
/iSp18//iSp18/*T*T*C*T*G*G*C*G*G*G*G*A*A*G*T
(SEQ ID NO: 58)
/iSp18//iSp18/*C*T*C*C*T*A*T*T*G*G*G*G*G*T*T*T*C*
C*T*A*T
(SEQ ID NO: 59)
/iSp18//iSp18/*A*C*C*C*C*C*T*C*T*A*C*C*C*C*C*T*C*
T*A*C*C*C*C*T*C*T
(SEQ ID NO: 60)
/iSp18//iSp18/*C*C*T*G*G*A*T*G*G*G*A*A
(SEQ ID NO: 61)
TTAGGGTTAGGGTTAGGGTTAGGG
(SEQ ID NO: 61)
T*T*A*G*G*G*T*T*A*G*G*G*T*T*A*G*G*G*T*T*A*G*G*G
(SEQ ID NO: 62)
TTAGGGTTAGGGTTAGGGTTAGGG/iSp18//iSp18//
(SEQ ID NO: 62)
T*T*A*G*G*G*T*T*A*G*G*G*T*T*A*G*G*G*T*T*A*G*G*G*/
iSp18//iSp18//
(SEQ ID NO: 63)
/iSp18//iSp18/TTAGGGTTAGGGTTAGGGTTAGGG
(SEQ ID NO: 63)
/iSp18//iSp18/*T*T*A*G*G*G*T*T*A*G*G*G*T*T*A*G*G*
G*T*T*A*G*G*G
(SEQ ID NO: 64)
CTATCTGUCGTTCTCTGU
(SEQ ID NO: 64)
C*T*A*T*C*T*G*U*C*G*T*T*C*T*C*T*G*U
(SEQ ID NO: 65)
CTATCTGUCGTTCTCTGU/iSp18//iSp18//
(SEQ ID NO: 65)
C*T*A*T*C*T*G*U*C*G*T*T*C*T*C*T*G*U*/iSp18//
iSp18//
(SEQ ID NO: 66)
/iSp18//iSp18/CTATCTGUCGTTCTCTGU
(SEQ ID NO: 66)
/iSp18//iSp18/*C*T*A*T*C*T*G*U*C*G*T*T*C*T*C*T*G*
U
(SEQ ID NO: 63)
/iSp18//iSp18/TTAGGGTTAGGGTTAGGGTTAGGG
(SEQ ID NO: 63)
/iSp18//iSp18/T*T*A*G*G*G*T*T*A*G*G*G*T*T*A*G*G*
G*T*T*A*G*G*G*
(SEQ ID NO: 66)
/iSp18//iSp18/CTATCTGUCGTTCTCTGU
/iSp18//iSp18/C*T*A*T*C*T*G*U*C*G*T*T*C*T*C*T*G*U*
(SEQ ID NO: 48)
/iSp18//iSp18/TGCTGGATGGGAA
(SEQ ID NO: 49)
/iSp18//iSp18/TGCCCTGGATGGGAA
(SEQ ID NO: 50)
/iSp18//iSp18/TGCTTGACACCTGGATGGGAA
(SEQ ID NO: 51)
/iSp18//iSp18/TCCTGAGCTTGAAGT
(SEQ ID NO: 52)
/iSp18//iSp18/TCCTGAGCTTGAAGT
(SEQ ID NO: 53)
/iSp18//iSp18/TTCTGGCGGGGAAGT
(SEQ ID NO: 54)
/iSp18//iSp18/CTCCTATTGGGGGTTTCCTAT
(SEQ ID NO: 55)
/iSp18//iSp18/ACCCCCTCTACCCCCTCTACCCCTCT
(SEQ ID NO: 56)
/iSp18//iSp18/CCTGGATGGGAA
(SEQ ID NO: 57)
/iSp18//iSp18/TTCTGGCGGGGAAGT
(SEQ ID NO: 58)
/iSp18//iSp18/CTCCTATTGGGGGTTTCCTAT
(SEQ ID NO: 59)
/iSp18//iSp18/ACCCCCTCTACCCCCTCTACCCCTCT
(SEQ ID NO: 56)
/iSp18//iSp18/CCTGGATGGGAA
(SEQ ID NO: 56)
/iSp18//iSp18/C*C*T*GGATGGGAA
(SEQ ID NO: 56)
/iSp18//iSp18/CCTGGATG*G*G*AA
(SEQ ID NO: 56)
/iSp18//iSp18/C*C*T*GGATG*G*G*AA
(SEQ ID NO: 67)
/iSp18//iSp18/CCTGGATGGGAA/Chol/
(SEQ ID NO: 68)
/iSp18//iSp18/CCTGGATGGGAA/Stryl/
and
(SEQ ID NO: 69)
/iSp18//iSp18/CCTGGATGGGAA/Palm/.

In some embodiments the antagonists of nucleic acid-interacting complexes are described in Kanzler, H. et al. Nature medicine 2007, 13, 552 and Banat, F. J.; et al. The Journal of experimental medicine 2005, 202, 1131, each of which is incorporated by reference.

The oligonucleotides may be linked to another compound such as a therapeutic or diagnostic compound. An exemplary therapeutic compound is an antigen. For instance, the oligonucleotides may be conjugated to a linker via the 5′ end or the 3′ end. E.g. [Sequence, 5′-3′]-Linker or Linker-[Sequence, 5′-3′] or via an internal nucleotide.

In other embodiments the oligonucleotide is an inhibitory nucleic acid. The oligonucleotide that is an inhibitory nucleic acid may be, for instance, an siRNA or an antisense molecule that inhibits expression of a protein that will have a therapeutic effect. The inhibitory nucleic acids may be designed using routine methods in the art.

An inhibitory nucleic acid typically causes specific gene knockdown, while avoiding off-target effects. Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA expression constructs) are used to reduce expression of a gene in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a specific promoter operably linked to a gene encoding the small interfering nucleic acid. In some cases, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006)). siRNAs with amide-linked oligoribonucleosides have been generated that are more resistant to S1 nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006).

Other molecules that can be used to inhibit expression of a gene include antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). The diverse array of suppression strategies that can be employed includes the use of DNA and/or RNA aptamers that can be selected to target a protein of interest.

Other inhibitor molecules that can be used include antisense nucleic acids (single or double stranded). Antisense nucleic acids include modified or unmodified RNA, DNA, or mixed polymer nucleic acids, and primarily function by specifically binding to matching sequences resulting in modulation of peptide synthesis (Wu-Pong, November 1994, BioPharm, 20-33). Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

An inhibitory nucleic acid useful in the invention will generally be designed to have partial or complete complementarity with one or more target genes. The target gene may be a gene derived from the cell, an endogenous gene, a transgene, or a gene of a pathogen which is present in the cell after infection thereof. Depending on the particular target gene, the nature of the inhibitory nucleic acid and the level of expression of inhibitory nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. As an example, the efficiency of inhibition may be determined by assessing the amount of gene product in the cell: mRNA may be detected with a hybridization probe having a nucleotide sequence outside the region used for the inhibitory nucleic acid, or translated polypeptide may be detected with an antibody raised against the polypeptide sequence of that region.

An expression enhancing oligonucleotide as used herein is a synthetic oligonucleotide that encodes a protein. The synthetic oligonucleotide may be delivered to a cell such that it is used by a cells machinery to produce a protein based on the sequence of the synthetic oligonucleotide. The synthetic oligonucleotide may be, for instance, synthetic DNA or synthetic RNA. “Synthetic RNA” refers to a RNA produced through an in vitro transcription reaction or through artificial (non-natural) chemical synthesis. In some embodiments, a synthetic RNA is an RNA transcript. In some embodiments, a synthetic RNA encodes a protein. In some embodiments, the synthetic RNA is a functional RNA. In some embodiments, a synthetic RNA comprises one or more modified nucleotides. In some embodiments, a synthetic RNA is up to 0.5 kilobases (kb), 1 kb, 1.5 kb, 2 kb, 2.5 kb, 3 kb, 4 kb, 5 kb, 6 kb, 7 kb, 8 kb, 9 kb, 10 kb, 15 kb, 20 kb, 25 kb, 30 kb or more in length. In some embodiments, a synthetic RNA is in a range of 0.1 kb to 1 kb, 0.5 kb to 2 kb, 0.5 kb to 10 kb, 1 kb to 5 kb, 2 kb to 5 kb, 1 kb to 10 kb, 3 kb to 10 kb, 5 kb to 15 kb, or 1 kb to 30 kb in length.

A diagnostic oligonucleotide is an oligonucleotide that interacts with a cellular marker to identify the presence of the marker in a cell or subject. Diagnostic oligonucleotides are well known in the art and typically include a label or are otherwise detectable.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis). An oligonucleotide of the nanostructure can be single stranded or double stranded. A double stranded oligonucleotide is also referred to herein as a duplex. Double-stranded oligonucleotides of the invention can comprise two separate complementary nucleic acid strands.

The nucleic acids useful in the nanostructures of the invention are synthetic or isolated nucleic acids.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded oligonucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

Oligonucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, polynucleotides associated with the invention may only contain modified or unmodified sugar moieties, while in other instances, polynucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′—OH group replaced by an H, alkoxy (or OR), R or alkyl, halogen, SH, SR, amino (such as NH₂, NHR, NR₂), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing undesirable cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. The sugar moiety can also be a hexose.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In some embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. The term “alkenyl” includes unsaturated aliphatic groups analogous in length and possible substitution to the alkyls described above, but that contain at least one double bond. The term alkenyl includes both “unsubstituted alkenyls” and “substituted alkenyls,” the latter of which refers to alkenyl moieties having independently selected substituents replacing a hydrogen on one or more carbons of the hydrocarbon backbone.

The term “hydrophobic modifications’ refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; and naphthyl.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, polynucleotides of the invention comprise 3′ and 5′ termini (except for circular oligonucleotides). The 3′ and 5′ termini of a polynucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

The nanostructures of the invention contemplate the use of linkers. The linkers may be linkers between nucleic acids, including standard phosphodiester internucleotide linkages as well as modified internucleotide linkages. The linkers may also be non-standard nucleotidic linkages that link nucleic acids with other nucleic acids or with other compounds such as proteins or G-quadruplex stabilizing domains. As used herein, the term nucleotide linkage includes a naturally occurring, unmodified phosphodiester moiety (—O—(PO²⁻)—O—) that covalently couples adjacent nucleomonomers as well as any analog or derivative of the native phosphodiester group that covalently couples adjacent nucleomonomers. Analogs or derivatives include phosphodiester analogs, e.g., phosphorothioate, phosphorodithioate, and P-ethyoxyphosphodiester, P-ethoxyphosphodiester, P-alkyloxyphosphotriester, methylphosphonate, phosphoramidates, thio-phosphoramidates, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47).

A non-nucleotidic linker or spacer sequence may be a peptide, a lipid, a polymer or an oligoethylene. Examples of linkers or spacers of the invention include HEG and PEG.

The surface density of the oligonucleotides in the nanostructure may depend on the size and type of nanostructure and on the length, sequence and concentration of the oligonucleotides. A surface density adequate to make the nanostructure stable and the conditions necessary to obtain it for a desired combination of nanostructure and oligonucleotides can be determined empirically. Generally, a surface density of at least 10 picomoles/cm will be adequate to provide stable nanostructure-oligonucleotide conjugates. Preferably, the surface density is at least 15 picomoles/cm. Since the ability of the oligonucleotides of the conjugates to hybridize with targets may be diminished if the surface density is too great, the surface density optionally is no greater than about 35-40 picomoles/cm². Methods are also provided wherein the oligonucleotide is bound to the nanoparticle at a surface density of at least 10 pmol/cm², at least 15 pmol/cm², at least 20 pmol/cm², at least 25 pmol/cm², at least 30 pmol/cm², at least 35 pmol/cm², at least 40 pmol/cm², at least 45 pmol/cm, at least 50 pmol/cm², or 50 pmol/cm² or more.

As used herein, the nano structure is a construct having an average diameter on the order of nanometers (i.e., between about 1 nm and about 1 micrometer. For example, in some instances, the diameter of the nanoparticle is from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, about 1 nm to about 20 nm in mean diameter, about 1 nm to about 10 nm in mean diameter, about 5 nm to about 150 nm in mean diameter, about 5 to about 50 nm in mean diameter, about 10 to about 30 nm in mean diameter, about 10 to 150 nm in mean diameter, about 10 to about 100 nm in mean diameter, about 10 to about 50 nm in mean diameter, about 30 to about 100 nm in mean diameter, or about 40 to about 80 nm in mean diameter.

Exemplary G-quadruplex forming nucleic acid-G-quadruplex stabilizing domain complexes include the following (“L” is a lipid group, including Palm-group):

| | | SEQ ID |
|---|---|---|
| 5′-Oligonucleotide-3′ | Type | NO: |
| TAGGGTTAGACAA | all-NP | 70 |
| Palm-TAGGGTTAGACAA | all-NP | 71 |
| Chol-TAGGGTTAGACAA | all-NP | 72 |
| TAGGGTTAGACAA | all-NPS | 70 |
| Palm-TAGGGTTAGACAA | all-NPS | 73 |
| Palm-TAGGGTTAGACAA | alt-NP/NPS | 74 |
| Palm-r-(TAGGGTTAGACAA) | all-NPS | 75 |
| TAGGGTTAGACAA | all-PS | 70 |
| Chol-TAGGGTTAGACAA | all-PS | 76 |
| TAGGGTTAGAC^{C18}AA | all-NPS | 77 |
| TAGGGTTAGACAA_{C20} | all-NPS | 78 |
| TAGGGTTAGACAA_{AEG-Palm} | all-NPS | 79 |
| Palm-(CCCTAA)₂ | all-NPS | 80 |
| Palm-(CCCTAA)₃ | all-NPS | 81 |
| (TTAGGG)₄ | all-PO | 82 |
| (TTAGGG)₄ | all-PS | 82 |
| Palm-(TTAGGG)₄ | all-NP | 83 |
| Palm-(TTAGGG)₄ | all-NPS | 83 |
| (TTAGGG)₃ | all-PS | 84 |
| (TTAGGG)₃ | all-NPS | 84 |
| Palm-(TTAGGG)₃ | all-NP | 85 |
| Palm-(TTAGGG)₃ | all-NPS | 85 |
| GGTTGGTGTGGTTGG* | all-PO | 86 |
| GGTTGGTGTGGTTGG | all-PS | 86 |
| GGTTGGTGTGGTTGG | all-NP | 86 |
| Palm-GGTTGGTGTGGTTGG | all-NP | 87 |
| Palm-GGTTGGTGTGGTTGG | all-NPS | 87 |
| L-GGTGGTGGTGGTTGTGGTGGTGGTGG | all-NPS | 93 |
| L-(GGGC*)₄ | all-NP | 94 |
| L-TTGGGGTT | all-NPS | |

Aspects of the invention relate to delivery of nanostructures to a subject for therapeutic and/or diagnostic use. The nanostructure may be administered alone or in any appropriate pharmaceutical carrier, such as a liquid, for example saline, or a powder, for administration in vivo. They can also be co-delivered with larger carrier particles or within administration devices. The nanostructure may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments, nanostructures associated with the invention are mixed with a substance such as a lotion (for example, aquaphor) and are administered to the skin of a subject, whereby the nanostructures are delivered through the skin of the subject. It should be appreciated that any method of delivery of nanoparticles known in the art may be compatible with aspects of the invention.

For use in therapy, an effective amount of the nanostructure can be administered to a subject by any mode that delivers the nanostructure to the desired cell. Administering pharmaceutical compositions may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

Thus, the invention in one aspect involves the finding that the nanostructures described herein are highly effective in mediating immune stimulatory and inhibitory effects. The agonist or antagonist oligonucleotides are useful therapeutically and prophylactically for stimulating the immune system to treat cancer, infectious diseases, allergy, asthma, autoimmune disease, and other disorders and to help protect against opportunistic infections following cancer chemotherapy. The strong yet balanced, cellular and humoral immune responses that result from, for example, TLR agonist stimulation reflect the body's own natural defense system against invading pathogens and cancerous cells.

Thus the nanostructure is useful in some aspects of the invention as a vaccine for the treatment of a subject at risk of developing or a subject having allergy or asthma, an infection with an infectious organism or a cancer in which a specific cancer antigen has been identified. The nanostructure can also be given without the antigen or allergen for protection against infection, allergy or cancer, and in this case repeated doses may allow longer term protection. A subject at risk as used herein is a subject who has any risk of exposure to an infection causing pathogen or a cancer or an allergen or a risk of developing cancer. For instance, a subject at risk may be a subject who is planning to travel to an area where a particular type of infectious agent is found or it may be a subject who through lifestyle or medical procedures is exposed to bodily fluids which may contain infectious organisms or directly to the organism or even any subject living in an area where an infectious organism or an allergen has been identified. Subjects at risk of developing infection also include general populations to which a medical agency recommends vaccination with a particular infectious organism antigen. If the antigen is an allergen and the subject develops allergic responses to that particular antigen and the subject may be exposed to the antigen, i.e., during pollen season, then that subject is at risk of exposure to the antigen.

A subject having an infection is a subject that has been exposed to an infectious pathogen and has acute or chronic detectable levels of the pathogen in the body. The nanostructure having immunostimulatory oligonucleotides can be used with or without an antigen to mount an antigen specific systemic or mucosal immune response that is capable of reducing the level of or eradicating the infectious pathogen. An infectious disease, as used herein, is a disease arising from the presence of a foreign microorganism in the body. It is particularly important to develop effective vaccine strategies and treatments to protect the body's mucosal surfaces, which are the primary site of pathogenic entry.

A subject having an allergy is a subject that has or is at risk of developing an allergic reaction in response to an allergen. An allergy refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions.

A subject having a cancer is a subject that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

A subject shall mean a human or vertebrate animal including but not limited to a dog, cat, horse, cow, pig, sheep, goat, turkey, chicken, primate, e.g., monkey, and fish (aquaculture species), e.g. salmon. Thus, the compounds described herein can also be used to treat cancer and tumors, autoimmune disease, infections, and allergy/asthma in human and non-human subjects.

As used herein, the term treat, treated, or treating when used with respect to an disorder such as an infectious disease, cancer, allergy, autoimmune disease or asthma refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

An antigen as used herein is a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and muticellular organisms such as parasites and allergens. The term antigen broadly includes any type of molecule which is recognized by a host immune system as being foreign. Antigens include but are not limited to cancer antigens, microbial antigens, and allergens.

A cancer antigen as used herein is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A microbial antigen as used herein is an antigen of a microorganism and includes but is not limited to virus, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

The nanostructures of the invention may also be coated with, linked to or administered in conjunction with an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

As used herein, the terms “cancer antigen” and “tumor antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

The nanostructures are also useful for treating and preventing autoimmune disease. Autoimmune disease is a class of diseases in which an subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer. Thus, in some aspects of the invention aimed at treating autoimmune disorders it is not recommended that the CpG immunostimulatory nucleic acids be administered with self antigens, particularly those that are the targets of the autoimmune disorder.

In other instances, the nanostructure may be delivered with low doses of self-antigens. A number of animal studies have demonstrated that mucosal administration of low doses of antigen can result in a state of immune hyporesponsiveness or “tolerance.” The active mechanism appears to be a cytokine-mediated immune deviation away from a Th1 towards a predominantly Th2 and Th3 (i.e., TGF-β dominated) response. The active suppression with low dose antigen delivery can also suppress an unrelated immune response (bystander suppression) which is of considerable interest in the therapy of autoimmune diseases, for example, rheumatoid arthritis and SLE. Bystander suppression involves the secretion of Th1-counter-regulatory, suppressor cytokines in the local environment where proinflammatory and Th1 cytokines are released in either an antigen-specific or antigen-nonspecific manner. “Tolerance” as used herein is used to refer to this phenomenon. Indeed, oral tolerance has been effective in the treatment of a number of autoimmune diseases in animals including: experimental autoimmune encephalomyelitis (EAE), experimental autoimmune myasthenia gravis, collagen-induced arthritis (CIA), and insulin-dependent diabetes mellitus. In these models, the prevention and suppression of autoimmune disease is associated with a shift in antigen-specific humoral and cellular responses from a Th1 to Th2/Th3 response.

In another aspect, the present invention is directed to a kit including one or more of the compositions previously discussed. A “kit,” as used herein, typically defines a package or an assembly including one or more of the compositions of the invention, and/or other compositions associated with the invention, for example, as previously described. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions that may be associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

In some embodiments, a kit associated with the invention includes one or more nanostructure components of the invention, such as a G-quadruplex nucleic acid, a therapeutic oligonucleotide, and a G-quadruplex stabilizing agent. A kit can also include one or more antigens.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the use of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoting” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A stable self-assembling nucleic acid nanostructure, comprising
a plurality of oligonucleotides, wherein each internucleotide linkage of the oligonucleotide is not a phosphorothioate linkage,
a plurality of G-quadruplex forming nucleic acids linked to the plurality of oligonucleotides, wherein the G-quadruplex forming nucleic acids is not TAGGGTT, and
a plurality of G-quadruplex stabilizing domains linked to the G-quadruplex forming nucleic acids,
wherein the oligonucleotides, the G-quadruplex forming nucleic acids and the G-quadruplex stabilizing domains form a plurality of G-quad structures.

2. A stable self-assembling nucleic acid nanostructure, comprising
a plurality of oligonucleotides,
a plurality of G-quadruplex forming nucleic acids linked to the plurality of oligonucleotides, wherein the G-quadruplex forming nucleic acids is not TAGGGTT, and
a plurality of G-quadruplex stabilizing domains linked to the G-quadruplex forming nucleic acids,
wherein when at least one of the G-quadruplex forming nucleic acids comprises GG, GGG, or GGGG and the oligonucleotide is CpG oligonucleotide the lipid is not diacyl lipid, wherein the oligonucleotides, the G-quadruplex forming nucleic acids and the G-quadruplex stabilizing domains form a plurality of G-quad structures.

3. The nanostructure of claim 1, wherein the self-assembling nucleic acid nanostructure does not have an inorganic core

4. The nanostructure of claim 1, wherein the plurality of oligonucleotides comprises oligonucleotides having identical nucleotide sequences or having at least two different nucleotide sequences.

5. The nanostructure of claim 1, wherein the plurality of oligonucleotides comprises oligonucleotides having at 2-10 different nucleotide sequences.

6. The nanostructure of claim 1, wherein the plurality of G-quadruplex forming nucleic acids comprise G-quadruplex forming nucleic acids having identical nucleotide sequences.

7. The nanostructure of claim 1, wherein the plurality of G-quadruplex forming nucleic acids comprises G-quadruplex forming nucleic acids having at least two different nucleotide sequences.

8. The nanostructure of claim 1, wherein the plurality of G-quadruplex stabilizing domains comprises identical G-quadruplex stabilizing domains.

9. The nanostructure of claim 1, wherein the plurality of G-quadruplex stabilizing domains comprises at least two different G-quadruplex stabilizing domains.

10. The nanostructure of claim 2, wherein each internucleotide linkage of the oligonucleotide is not a phosphorothioate linkage.

11. The nanostructure of claim 2, wherein each internucleotide linkage of the oligonucleotide is a phosphorothioate linkage.

12. The nanostructure of claim 1, wherein at least one internucleotide linkage of the G-quadruplex forming nucleic acid is selected from a N3′-P5′ phosphoramidate linkage and a N3′-P5′thio-phosphoramidate linkage.

13. The nanostructure of claim 1, wherein each internucleotide linkage of the oligonucleotide is selected from a N3′-P5′phosphoramidate linkage and a N3′-P5′thio-phosphoramidate linkage.

14. The nanostructure of claim 1, wherein each internucleotide linkage of the G-quadruplex forming nucleic acid is selected from a N3′-P5′phosphoramidate linkage and a N3′-P5′thio-phosphoramidate linkage.

15. The nanostructure of claim 1, wherein the thermodynamic stability of the nanostructure is high enough to provide for the overall structural stability of constructs under physiological salt and temperature conditions.

16. The nanostructure of claim 1, wherein at least one of the oligonucleotides have 5′ termini exposed to the outside surface of the nanostructure.

17. The nanostructure of claim 1, wherein the plurality of oligonucleotides comprises CpG oligonucleotides.

18. The nanostructure of claim 17, wherein the CpG oligonucleotides are selected from the group consisting of A-class, B-class and C-class CpG oligonucleotides.

19. The nanostructure of claim 1, wherein the plurality of oligonucleotides comprises RNA or antisense oligonucleotides.

20. The nanostructure of claim 1, wherein the plurality of oligonucleotides comprises TLR7 antagonists.

21. The nanostructure of claim 1, wherein the plurality of oligonucleotides comprises TLR8 antagonists.

22. The nanostructure of claim 1, wherein the plurality of oligonucleotides comprises TLR9 antagonists.

23. The nanostructure of claim 1, wherein the plurality of oligonucleotides comprises TLR7 agonists.

24. The nanostructure of claim 1, wherein the plurality of oligonucleotides comprises TLR8 agonists.

25. The nanostructure of claim 1, wherein the plurality of oligonucleotides comprises TLR9 agonists.

26-34. (canceled)

35. A method for delivering a plurality of oligonucleotides to a subject, comprising
administering to a subject a stable self-assembling nucleic acid nanostructure, comprising
a plurality of oligonucleotides, a plurality of G-quadruplex forming nucleic acids linked to the plurality of oligonucleotides, and a plurality of G-quadruplex stabilizing domains linked to the G-quadruplex forming nucleic acids, wherein the oligonucleotides, the G-quadruplex forming nucleic acids and the G-quadruplex stabilizing domains form a plurality of G-quad structures, and
wherein the plurality of oligonucleotides is delivered to the subject.

36-69. (canceled)


SEQUENCE LISTING (94 sequences)

SEQ ID NO: 1 - DNA, 15 bp, Artificial Sequence
  Synthetic Polynucleotide
ggttggtgtggttgg

SEQ ID NO: 2 - DNA, 10 bp, Artificial Sequence
  Synthetic Polynucleotide
gggttttggg

SEQ ID NO: 3 - DNA, 26 bp, Artificial Sequence
  Synthetic Polynucleotide
ggtggtggtggttgtggtggtggtgg

SEQ ID NO: 4 - RNA, 18 nt, Artificial Sequence
  Synthetic Polynucleotide
ccgucuguugugugacuc

SEQ ID NO: 5 - RNA, 20 nt, Artificial Sequence
  Synthetic Polynucleotide
gccaccgagccgaaggcacc

SEQ ID NO: 6 - RNA, 20 nt, Artificial Sequence
  Synthetic Polynucleotide
uauauauauauauauauaua

SEQ ID NO: 7 - RNA, 20 nt, Artificial Sequence
  Synthetic Polynucleotide
uuauuauuauuauuauuauu

SEQ ID NO: 8 - RNA, 20 nt, Artificial Sequence
  Synthetic Polynucleotide
uuuuauuuuauuuuauuuua

SEQ ID NO: 9 - RNA, 20 nt, Artificial Sequence
  Synthetic Polynucleotide
ugugugugugugugugugug

SEQ ID NO: 10 - RNA, 20 nt, Artificial Sequence
  Synthetic Polynucleotide
uuguuguuguuguuguuguu

SEQ ID NO: 11 - RNA, 20 nt, Artificial Sequence
  Synthetic Polynucleotide
uuuguuuguuuguuuguuug

SEQ ID NO: 12 - RNA, 24 nt, Artificial Sequence
  Synthetic Polynucleotide
uuauuuauuuauuuauuuauuuau

SEQ ID NO: 13 - RNA, 24 nt, Artificial Sequence
  Synthetic Polynucleotide
uuguuuguuuguuuguuuguuugu

SEQ ID NO: 14 - RNA, 20 nt, Artificial Sequence
  Synthetic Polynucleotide
gcccgucuguugugugacuc

SEQ ID NO: 15 - RNA, 18 nt, Artificial Sequence
  Synthetic Polynucleotide
guccuucaaguccuucaa

SEQ ID NO: 16 - DNA, 20 bp, Artificial Sequence
  Synthetic Polynucleotide
ggtgcatcgatgcagggggg

SEQ ID NO: 17 - DNA, 22 bp, Artificial Sequence
  Synthetic Polynucleotide
tccatggacgttcctgagcgtt

SEQ ID NO: 18 - DNA, 22 bp, Artificial Sequence
  Synthetic Polynucleotide
tcgtcgttcgaacgacgttgat

SEQ ID NO: 19 - DNA, 22 bp, Artificial Sequence
  Synthetic Polynucleotide
tcgtcgacgatccgcgcgcgcg

SEQ ID NO: 20 - DNA, 20 bp, Artificial Sequence
  Synthetic Polynucleotide
ggggtcaacgttgagggggg

SEQ ID NO: 21 - DNA, 24 bp, Artificial Sequence
  Synthetic Polynucleotide
tcgtcgttttgtcgttttgtcgtt

SEQ ID NO: 22 - DNA, 22 bp, Artificial Sequence
  Synthetic Polynucleotide
tcgtcgttgtcgttttgtcgtt

SEQ ID NO: 23 - DNA, 20 bp, Artificial Sequence
  Synthetic Polynucleotide
gggggacgatcgtcgggggg

SEQ ID NO: 24 - DNA, 21 bp, Artificial Sequence
  Synthetic Polynucleotide
ggggacgacgtcgtggggggg

SEQ ID NO: 25 - DNA, 22 bp, Artificial Sequence
  Synthetic Polynucleotide
tcgtcgttttcggcgcgcgccg

SEQ ID NO: 26 - DNA, 25 bp, Artificial Sequence
  Synthetic Polynucleotide
tcgtcgtcgttcgaacgacgttgat

SEQ ID NO: 27 - DNA, 15 bp, Artificial Sequence
  Synthetic Polynucleotide
tcctggaggggttgt

SEQ ID NO: 28 - DNA, 18 bp, Artificial Sequence
  Synthetic Polynucleotide
tgctcctggaggggttgt

SEQ ID NO: 29 - DNA, 13 bp, Artificial Sequence
  Synthetic Polynucleotide
tgctggatgggaa

SEQ ID NO: 30 - DNA, 15 bp, Artificial Sequence
  Synthetic Polynucleotide
tgccctggatgggaa

SEQ ID NO: 31 - DNA, 21 bp, Artificial Sequence
  Synthetic Polynucleotide
tgcttgacacctggatgggaa

SEQ ID NO: 32 - DNA, 13 bp, Artificial Sequence
  Synthetic Polynucleotide
tgctggatgggaa

SEQ ID NO: 33 - DNA, 15 bp, Artificial Sequence
  Synthetic Polynucleotide
tgccctggatgggaa

SEQ ID NO: 34 - DNA, 21 bp, Artificial Sequence
  Synthetic Polynucleotide
tgcttgacacctggatgggaa

SEQ ID NO: 35 - DNA, 15 bp, Artificial Sequence
  Synthetic Polynucleotide
tcctgagcttgaagt

SEQ ID NO: 36 - DNA, 15 bp, Artificial Sequence
  Synthetic Polynucleotide
tcctgagcttgaagt

SEQ ID NO: 37 - DNA, 15 bp, Artificial Sequence
  Synthetic Polynucleotide
ttctggcggggaagt

SEQ ID NO: 38 - DNA, 21 bp, Artificial Sequence
  Synthetic Polynucleotide
ctcctattgggggtttcctat

SEQ ID NO: 39 - DNA, 26 bp, Artificial Sequence
  Synthetic Polynucleotide
accccctctaccccctctacccctct

SEQ ID NO: 40 - DNA, 12 bp, Artificial Sequence
  Synthetic Polynucleotide
cctggatgggaa

SEQ ID NO: 41 - DNA, 15 bp, Artificial Sequence
  Synthetic Polynucleotide
ttctggcggggaagt

SEQ ID NO: 42 - DNA, 21 bp, Artificial Sequence
  Synthetic Polynucleotide
ctcctattgggggtttcctat

SEQ ID NO: 43 - DNA, 26 bp, Artificial Sequence
  Synthetic Polynucleotide
accccctctaccccctctacccctct

SEQ ID NO: 44 - DNA, 12 bp, Artificial Sequence
  Synthetic Polynucleotide
cctggatgggaa

SEQ ID NO: 45 - DNA, 12 bp, Artificial Sequence
  Synthetic Polynucleotide
cctggatgggaa

SEQ ID NO: 46 - DNA, 12 bp, Artificial Sequence
  Synthetic Polynucleotide
cctggatgggaa

SEQ ID NO: 47 - DNA, 12 bp, Artificial Sequence
  Synthetic Polynucleotide
cctggatgggaa

SEQ ID NO: 48 - DNA, 13 bp, Artificial Sequence
  Synthetic Polynucleotide
tgctggatgggaa

SEQ ID NO: 49 - DNA, 15 bp, Artificial Sequence
  Synthetic Polynucleotide
tgccctggatgggaa

SEQ ID NO: 50 - DNA, 21 bp, Artificial Sequence
  Synthetic Polynucleotide
tgcttgacacctggatgggaa

SEQ ID NO: 51 - DNA, 15 bp, Artificial Sequence
  Synthetic Polynucleotide
tcctgagcttgaagt

SEQ ID NO: 52 - DNA, 15 bp, Artificial Sequence
  Synthetic Polynucleotide
tcctgagcttgaagt

SEQ ID NO: 53 - DNA, 15 bp, Artificial Sequence
  Synthetic Polynucleotide
ttctggcggggaagt

SEQ ID NO: 54 - DNA, 21 bp, Artificial Sequence
  Synthetic Polynucleotide
ctcctattgggggtttcctat

SEQ ID NO: 55 - DNA, 26 bp, Artificial Sequence
  Synthetic Polynucleotide
accccctctaccccctctacccctct

SEQ ID NO: 56 - DNA, 12 bp, Artificial Sequence
  Synthetic Polynucleotide
cctggatgggaa

SEQ ID NO: 57 - DNA, 15 bp, Artificial Sequence
  Synthetic Polynucleotide
ttctggcggggaagt

SEQ ID NO: 58 - DNA, 21 bp, Artificial Sequence
  Synthetic Polynucleotide
ctcctattgggggtttcctat

SEQ ID NO: 59 - DNA, 26 bp, Artificial Sequence
  Synthetic Polynucleotide
accccctctaccccctctacccctct

SEQ ID NO: 60 - DNA, 12 bp, Artificial Sequence
  Synthetic Polynucleotide
cctggatgggaa

SEQ ID NO: 61 - DNA, 24 bp, Artificial Sequence
  Synthetic Polynucleotide
ttagggttagggttagggttaggg

SEQ ID NO: 62 - DNA, 24 bp, Artificial Sequence
  Synthetic Polynucleotide
ttagggttagggttagggttaggg

SEQ ID NO: 63 - DNA, 24 bp, Artificial Sequence
  Synthetic Polynucleotide
ttagggttagggttagggttaggg

SEQ ID NO: 64 - DNA, 18 bp, Artificial Sequence
  Synthetic Polynucleotide
ctatctgucgttctctgu

SEQ ID NO: 65 - DNA, 18 bp, Artificial Sequence
  Synthetic Polynucleotide
ctatctgucgttctctgu

SEQ ID NO: 66 - DNA, 18 bp, Artificial Sequence
  Synthetic Polynucleotide
ctatctgucgttctctgu

SEQ ID NO: 67 - DNA, 12 bp, Artificial Sequence
  Synthetic Polynucleotide
cctggatgggaa

SEQ ID NO: 68 - DNA, 12 bp, Artificial Sequence
  Synthetic Polynucleotide
cctggatgggaa

SEQ ID NO: 69 - DNA, 12 bp, Artificial Sequence
  Synthetic Polynucleotide
cctggatgggaa

SEQ ID NO: 70 - DNA, 13 bp, Artificial Sequence
  Synthetic Polynucleotide
tagggttagacaa

SEQ ID NO: 71 - DNA, 13 bp, Artificial Sequence
  Synthetic Polynucleotide
tagggttagacaa

SEQ ID NO: 72 - DNA, 13 bp, Artificial Sequence
  Synthetic Polynucleotide
tagggttagacaa

SEQ ID NO: 73 - DNA, 13 bp, Artificial Sequence
  Synthetic Polynucleotide
tagggttagacaa

SEQ ID NO: 74 - DNA, 13 bp, Artificial Sequence
  Synthetic Polynucleotide
tagggttagacaa

SEQ ID NO: 75 - DNA, 13 bp, Artificial Sequence
  Synthetic Polynucleotide
tagggttagacaa

SEQ ID NO: 76 - DNA, 13 bp, Artificial Sequence
  Synthetic Polynucleotide
tagggttagacaa

SEQ ID NO: 77 - DNA, 13 bp, Artificial Sequence
  Synthetic Polynucleotide
tagggttagacaa

SEQ ID NO: 78 - DNA, 13 bp, Artificial Sequence
  Synthetic Polynucleotide
tagggttagacaa

SEQ ID NO: 79 - DNA, 13 bp, Artificial Sequence
  Synthetic Polynucleotide
tagggttagacaa

SEQ ID NO: 80 - DNA, 12 bp, Artificial Sequence
  Synthetic Polynucleotide
ccctaaccctaa

SEQ ID NO: 81 - DNA, 18 bp, Artificial Sequence
  Synthetic Polynucleotide
ccctaaccctaaccctaa

SEQ ID NO: 82 - DNA, 24 bp, Artificial Sequence
  Synthetic Polynucleotide
ttagggttagggttagggttaggg

SEQ ID NO: 83 - DNA, 24 bp, Artificial Sequence
  Synthetic Polynucleotide
ttagggttagggttagggttaggg

SEQ ID NO: 84 - DNA, 18 bp, Artificial Sequence
  Synthetic Polynucleotide
ttagggttagggttaggg

SEQ ID NO: 85 - DNA, 18 bp, Artificial Sequence
  Synthetic Polynucleotide
ttagggttagggttaggg

SEQ ID NO: 86 - DNA, 15 bp, Artificial Sequence
  Synthetic Polynucleotide
ggttggtgtggttgg

SEQ ID NO: 87 - DNA, 15 bp, Artificial Sequence
  Synthetic Polynucleotide
ggttggtgtggttgg

SEQ ID NO: 88 - DNA, 20 bp, Artificial Sequence
  Synthetic Polynucleotide
tccatgacgttcctgatgct

SEQ ID NO: 89 - DNA, 12 bp, Artificial Sequence
  Synthetic Polynucleotide
cctggatgggaa

SEQ ID NO: 90 - DNA, 13 bp, Artificial Sequence
  Synthetic Polynucleotide
tagggttagacaa

SEQ ID NO: 91 - DNA, 26 bp, Artificial Sequence
  Synthetic Polynucleotide
ggtggtggtggttgtggtggtggtgg

SEQ ID NO: 92 - DNA, 10 bp, Artificial Sequence
  Synthetic Polynucleotide
gggttttggg

SEQ ID NO: 93 - DNA, 26 bp, Artificial Sequence
  Synthetic Polynucleotide
ggtggtggtggttgtggtggtggtgg

SEQ ID NO: 94 - DNA, 16 bp, Artificial Sequence
  Synthetic Polynucleotide
gggcgggcgggcgggc